(19) United States
(12) Patent Application Publication
(10) Pub. No.: US 2012/0283235 A1
(43) Pub. Date: Nov. 8, 2012

(54) Dermatologic and Cosmetic Compositions

(75) Inventors: John P. McCook, Frisco, TX (US); Peter L. Dorogi, Easton, PA (US); David B. Vasily, Bethlehem, PA (US)

(73) Assignee: DISCOVERY PARTNERS LLC, Frisco, TX (US)

(21) Appl. No.: 13/502,709

(22) PCT Filed: Oct. 20, 2010
(86) PCT No.: PCT/US10/53430

§ 371 Date: Jul. 26, 2012

Related U.S. Application Data
(60) Provisional application No. 61/253,439, filed on Oct. 20, 2009.

(51) Int. Cl. A61K 8/49; A61Q 19/00; A61Q 19/08; A61K 8/58

(52) U.S. Cl. 514/185; 514/410

(57) ABSTRACT

Provided are compositions and methods for treatment of conditions and diseases of the skin, for example fine lines and wrinkles and rosacea. The compositions include a porphyrin that is a chlorophyllin, a chlorin compound, a complex of a chlorin compound, or a salt or ester of the chlorin compound or its complex.

### BACKGROUND OF THE INVENTION

Chlorophyllin, chlorin e4, chlorin e6, as well as their salts have been used in wound healing. However, applicants have surprisingly and unexpectedly found that topical administration of these compounds decreases collagen type 1.

U.S. Pat. No. 5,998,395 discloses methods of treating inflammatory dermatoses by combined application of a corticosteroid and a retinoid. Once or twice daily application of the combined corticosteroid-retinoid therapy is taught to be more effective than treatment with either active ingredient alone.

The treatment of photodamaged skin (i.e., exhibiting fine lines, wrinkles, uneven pigmentation) with combinations of Retinol and vitamin C (or its derivatives) is reported in the literature. See, e.g., Seité et al, Skin Pharmacol Physiol. Vol. 18, No. 2, pp. 81-87 (Mar-April 2005). However, it is also known, that treatment with retinol can cause skin irritation.

Dark circles and edema under the eyes have been associated with capillary leakage and fraying of collagen bundles. U.S. Pat. Nos. 5,643,587 and 6,607,735 discuss potential causes and mechanisms for puffiness or bagginess under the eyes. (To the extent pertinent, granted U.S. patents and published U.S. patent applications cited herein are incorporated by reference in their entirety.) Because the skin under the eyes is the thinnest in the human body it is also particularly susceptible to irritation from treatment with retinoids.

Dihydroxytestosterone (“DHT”) is an androgenic hormone that has been associated with hair loss. 5-alpha-reductase catalyses the production of DHT. One aspect of the present invention is directed to topical compositions comprising the chlorophyllin, ascorbate (or other vitamin C derivative), retinol and at least one androgen receptor inhibitor, particularly agents such as spironolactone that compete for DHT or otherwise block the binding of DHT to its receptors. See, J R Matias et al., J. Invest. Dermatol., Vol. 91, No. 5, pp. 429-433 (1988). See also, Berardesca et al., Intl J. Tissue Reactions, Vol. 10, No. 2, pp. 115-119 (1988); Akamatsuet al. J. Invest. Dermatol., Vol. 100, 660-662 (1993).

Zinc PCA, the zinc salt of L-Pyrrolidone Carboxylic Acid, has been reported to inhibit 5-alpha-reductase activity, and thereby regulate the activity of the sebaceous glands and reduce skin sebum

U.S. Pat. No. 7,025,955 discloses hair care compositions comprising panthenol, Zinc PCA, green tea extract and retinol.

U.S. Pat. No. 6,126,940 teaches stimulating hair growth by applying to the scalp a composition comprising proanthocyanadins in combination with anti-inflammatory agents (including dipotassium glycyrrhetinate) and antioxidants (including gallic acid and its propyl gallate ester).

### BRIEF DESCRIPTION OF THE FIGURES

FIG. 1 is a graphical representation of the results of testing collagen production during culturing of human dermal fibroblast cells in the presence of chlorophyllins complexes of the present invention.

### SUMMARY OF THE INVENTION

A first aspect of the present invention relates to combination topical therapies to treat and prevent photodamage, as expressed as facial fine lines and wrinkles and uneven pigmentation, including lentigines, wherein (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid.

A second aspect of the present invention is directed to methods and formulations for the topical treatment of dark circles under the eyes comprising topical administration of (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid.

A third aspect of the present invention is directed to methods and formulations for the topical treatment of acne comprising topical administration of (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid, preferably retinol.

A fourth aspect of the present invention is directed to methods and formulations for the topical treatment of inflammatory dermatologic conditions, including rosacea, comprising topical administration of (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid, preferably retinol.

A fifth aspect of the present invention is directed to methods and formulations for the topical treatment of thinning hair or alopecia comprising topical administration of (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid, preferably retinol and/or a 5-alpha reductase inhibitor.

A sixth aspect of the present invention relates to combination topical therapies to treat, brighten or lighten facial skin classified as Fitzpatrick Types III-VI (3-6) or to treat hyperpigmented facial or body skin with the condition of vitiligo wherein (i) chlorophyllin, chlorin e4, chlorin e6, or ethyl esters of chlorin e4 or e6, the chlorins or chlorin esters preferably in the form of the sodium or potassium salts of their copper and zinc complexes, is administered together with (ii) vitamin C or a vitamin C derivative, preferably ascorbate, more preferably tetrahexyldecylascorbate and, (iii) optionally a retinoid.

### DETAILED DESCRIPTION OF THE INVENTION

As used in the present application, by the phrase “safe and effective amount” is meant a sufficient amount of a compound or composition to induce a clinically positive modification in the condition being treated (based on clinical observation, clinical measurement and/or self-reporting) that does not cause significant side effects (e.g., significant skin irritation or sensitization). As will be appreciated by the person of skill in the art, a safe and effective amount of a compound or composition will vary among patients based on, among other things, the patient's skin type, age and health, as well as the severity of the condition and the duration of the treatment.

### Decreased Collagen Type I in Human Dermal Fibroblast Conditioned Media

Adult human dermal fibroblasts (48 year old, Caucasian, female facial cells, from Cell Applications, Inc. San Diego, Calif., Catalog #106-05A, Lot 1339) were incubated for seventy-two hours in Fibroblast Growth Serum (Cell Applications, cat. #116-500). Four test formulations, each formulation used at three final test sample dilutions of 2.5%, 0.5% and 0.05%, were added to the incubated cells. All concentrations expressed in weight/weight unless otherwise noted:
(i) Test Formulation #1, CHL-01-057 (labeled “1”) served as a control and contained 90% purified water and 10% butylene glycol;
(ii) Test Formulation #2, CHL-01-053 (labeled “2”) containing 0.2% of 1-Disodium Copper Chlorin E4 in 10% butylene glycol and 89.8% purified water;
(iii) Test Formulation #3, (labeled #3) CHL-01-0 51 containing 0.2% Sodium Copper Chlorophyllin complex, USP in 10% butylene glycol and 89.8% purified water;
(iv) Test Formulation #4, CHL-01-055 (labeled “4”) containing 0.2% Sodium Zinc Chlorophyllin in 10% butylene glycol and 89.8% purified water.

Based on the final dilutions, the amount of chlorin, chlorophyllin, or control compound added to the cell culture was 50 ppm, 10 ppm, and 1 ppm (ppm=parts per million by weight).

Each of the above formulations was prepared by adding butylene glycol to water and mixing on a heating plate with a magnetic stirrer until the glycol fully dissolved. The active ingredient in Test Formulation #s 2-4 was then added and mixed until dissolved.

The same human fibroblast dermal cells were also incubated in magnesium ascorbyl phosphate (100 ug/ml, “MAP”) and ascorbic acid (10 ug/ml, “Vit. C”) as positive controls. Sterile water was used as a negative control.

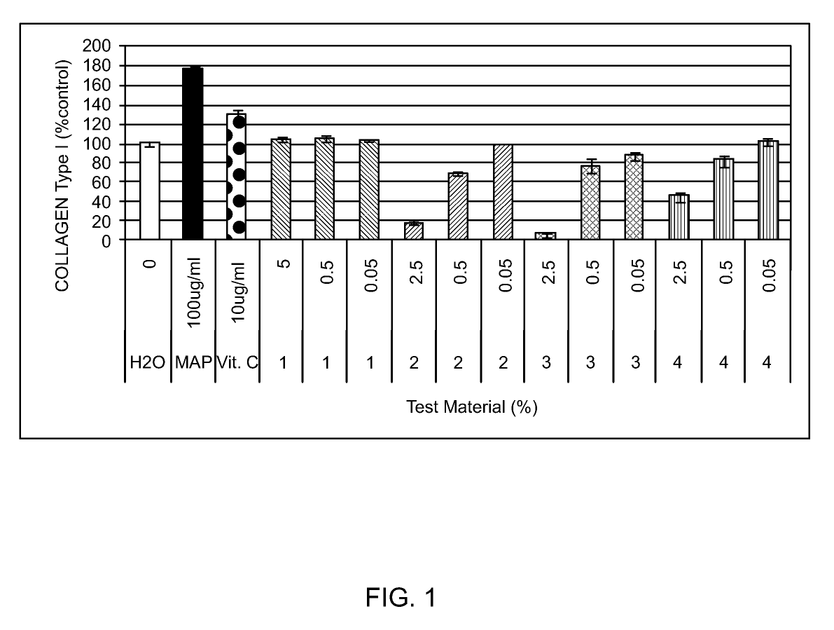

The test results are presented in FIG. 1. Surprisingly and unexpectedly, the test formulations containing chlorophyllin and chlorin materials did not stimulate the production of type 1 collagen. To the contrary, cells treated with these formulations showed a dose-dependent decrease in collagen activity. Cells cultured with the positive controls (vitamin C and Magnesium Ascorbyl Phosphate) showed a strong stimulatory activity, confirming the validity of the study design.

A first embodiment of the present invention provides methods for improving the cosmetic appearance of the skin in terms of reducing one or more cosmetic dermatologic parameters selected from the group of fine lines, coarse wrinkles (rhytids) and pleating, marionette lines, nasolabial folds, pore size, oiliness, dark circles, uneven pigmentation (in particular hyperpigmentation), redness (erythema), which methods include the step of applying one or more particular porphyrins. Preferred porphyrins include chlorophyllin, chlorin e4, chlorin e6, ethyl esters of chlorin e4 or e6 (collectively ‘the chlorins’), wherein the chlorins or chlorin ethyl esters are preferably in the form of their sodium or potassium salts of their copper or zinc complexes.

Chlorophyllin copper complex and chlorophyllin zinc complex and sodium salts thereof, commonly referred to as chlorophyllins, and their uses in topical formulations are described in U.S. Patent Application Publication 2008/0317836. As far as Applicants are aware, the porphyrins chlorine e4 and chlorine e6, their ethyl esters and/or salts, whether or not in the form of a Cu or Zn complexes, have not been used in dermatology without some type of adjunct therapy.

In a preferred embodiment of the present invention sodium chlorophyllin copper complex, sodium chlorophyllin zinc complex, or mixtures thereof are contained within a dispersion of liposomes (“liposomal dispersion”). Preferably, the liposomes have phospholipid shells, more preferably the phospholipid is derived from lecithin. In a particularly, preferred embodiment, the lecithin is derived from soybean or egg. In an even more preferred embodiment, the phospholipid shell contains phosphatidylcholine at a concentration of at least about 85% based on the total weight of the phospholipid shell. Additional materials suitable for forming the liposomal dispersion are described in U.S. Patent Application Publication 2008/0317836.

In a preferred embodiment the ratio of (i) chlorophyllin copper complex, chlorophyllin zinc complex, chlorin e4 or chlorin e6, chlorin e4 or chlorin e6 ethyl esters, and their salts, or mixtures thereof to (ii) phospholipid is about 1 to 2.

Preferably, the liposomal dispersion comprising the chlorophyllin copper complex, the chlorophyllin zinc complex, chlorin e4 or chlorin e6, chlorine e4 or chlorine e6 ethyl esters and their salts, or mixtures thereof, has a pH of from about 7.0 to 8.0, more preferably from 7.2 to 7.6.

The concentration at which chlorophyllin copper complex, chlorophyllin zinc complex, chlorin e4, e6, chlorine e4 ethyl ester, or e6 ethyl ester and/or their salts (or mixtures thereof) is present in a finished formulation will depend on whether the chlorophyllin and/or chlorin is delivered from the liposomal dispersion and whether that formulation is administered in combination with a second formulation (e.g., from a dual-chamber container, as described below).

In one preferred embodiment, a vitamin C derivative, preferably tetrahexadecyl ascorbate is simultaneously administered, preferably at a concentration of from about 0.5% to about 2%. In another preferred embodiment, the vitamin C derivative is sodium ascorbyl phosphate, preferably at a concentration of from about 1% to about 3%.

As used in the present invention, “retinoid” means natural and synthetic analogs of vitamin A, as well as geometric isomers and stereoisomers of these compounds, or compounds that exhibit structures and activities similar to vitamin A. Retinoids suitable for use in the present invention are selected from the group consisting of retinol, retinal, retinol esters (C₂-C₂₂ alkyl esters of retinol, including retinyl palmitate, retinyl acetate, retinyl propionate), retinoic acid (including all-trans retinoic acid and/or 13-cis-retinoic acid), tocopheryl-retinoate [tocopherol ester of retinoic acid (trans- or cis-), adapalene {6-[3-(1-adamantyl)-4-methoxyphenyl]-2-naphthoic acid}, tazarotene (ethyl 6-[2-(4,4-dimethylthiochroman-6-yl)-ethynyl]nicotinate as well as the retinoids described in U.S. Pat. Nos. 4,677,120; 4,885,311; 5,049,584; and 5,124,356.

In embodiments of the present invention in which the retinoid is retinoic acid, the retinoid is administered at a concentration of from about 0.05% to about 0.1%, preferably from about 0.01 to about 0.1%.

In embodiments of the present invention in which the retinoid is retinol, the retinoid is administered at a concentration of from about 0.125% to about 1.0%, preferably from about 0.25 to about 1.0%.

In particularly preferred embodiments, the retinoid is releaseably entrapped within solid spherical particles having an average diameter of about 1 micron to about 100 microns, having a continuous non-collapsible network of pores open to the exterior of the particles. Particles of this type are described in U.S. Pat. No. 5,955,109.

In an especially preferred embodiment, the method for reducing the appearance of one or more cosmetic dermatologic parameters selected from the group of fine lines, coarse wrinkles (rhytids) and pleating, marionette lines, nasolabial folds, pore size, oiliness, dark circles, uneven pigmentation (in particular hyperpigmentation), redness (erythema) comprises the administration of a single formulation containing both a retinoid and ascorbate (or other vitamin C derivative). In these especially preferred embodiments, the pH of the formulation is from about 4 to about 5.

In some preferred embodiments in which a retinoid is administered together with a chlorophyllin or chlorin compound, two compositions are administered, the first composition comprising a retinoid and the ascorbate (or other vitamin C derivative), the second composition containing the chlorophyllin complex and/or chlorin compound. In these preferred embodiments, the two compositions may be dispensed from a single container in which the two formulations are stored separately prior to dispense (a “dual-chamber container”). The dual-chamber container may have two separate actuators/pumps—each having an orifice for dispensing one of the two formulations. Alternatively, the dual-chamber container may contain two pumps and one actuator from which the two formulations are dispensed either through two orifices (e.g., side-by-side) or from a single common orifice. A non-limiting example of a dual-chamber container suitable for use in this embodiment of the invention is described in U.S. Pat. No. 6,462,025.

The first embodiment of the invention is illustrated Example 1 below. Other objects and advantages of this aspect of the present invention will become apparent and obvious from this study which is merely illustrative of the invention.

### Example 1

### Clinical Study

Preparation of Test Formulation A—Vitamin C+Retinol Lotion (0.25% (A1) and 0.5% (A2))

| | | 0.25% | 0.5% |
|---|---|---|---|
| | | Retinol | Retinol |
| Phase | Ingredient (INCI Name) | A1 | A2 |
| A | Purified Water | 64.589 | 63.339 |
| A | Carbomer | 0.500 | 0.500 |
| A | Xanthan Gum | 0.200 | 0.200 |
| A | Sodium Hyaluronate | 0.010 | 0.010 |
| A | Disodium EDTA | 0.100 | 0.100 |
| A | Glycerin | 4.000 | 4.000 |
| A | Aloe Barbadensis Leaf Juice | 1.000 | 1.000 |
| A | Allantoin | 0.300 | 0.300 |
| B | Ethylhexyl Stearate | 4.000 | 4.000 |
| B | Glyceryl Stearate, PEG-100 Stearate | 4.000 | 4.000 |
| B | Cetearyl Alcohol, Steareth-10, Steareth-20 | 2.000 | 2.000 |
| B | Caprylic/Capric Triglyceride | 5.000 | 5.000 |
| B | Dimethicone | 1.000 | 1.000 |
| B | PPG-12/SMDI Copolymer | 0.500 | 0.500 |
| B | ButylatedHydroxytoluene | 0.050 | 0.050 |
| B | Polyacrylamide, C13-14 Isoparaffin, Laureth-7 | 1.000 | 1.000 |
| C | Phenoxyethanol | 0.800 | 0.800 |
| C | Butylene Glycol, o-Cymen-5-OL | 0.500 | 0.500 |
| C | Tocopheryl Acetate | 0.500 | 0.500 |
| C | Cyclodextrin, Glycine Soja (Soybean) Germ | 0.100 | 0.100 |
| | Extract | | |
| C | Glycerin, Palmitoyl Tripeptide-5 | 3.000 | 3.000 |
| C | Tetrahexyldecyl Ascorbate | 0.500 | 0.500 |
| C | Glycerin, Water, Camellia Sinensis (Green | 0.100 | 0.100 |
| | Tea) Leaf Extract | | |
| D | Isopentyldiol | 4.000 | 4.000 |
| D | Glycerin | 1.000 | 1.000 |
| D | AllylMethacrylate Crosspolymer, Polysorbate | 1.250 | 2.500 |
| | 20, Retinol, BHT | | |
| D | Beta Carotene | 0.001 | 0.001 |

Assemble Phase A by first combining Carbomer and Xanthan Gum. Slowly add this mixture to the rest of Phase A while mixing with a propeller mixer. Heat Phase A to 55° C. Combine Phase B ingredients; heat to 55° C. Assemble Phases C and D separately. Slowly add Phase A into Phase B and immediately begin mixing with Silverson L4RT homogenizer with standard head at 7,000 rpm for 5-10 minutes. Add Phase C to A/B at room temperature (20-25° C.) and mix with Silverson homogenizer at 3,000 rpm for about 2 minutes or until uniform. Add Phase D to A/B/C at room temperature and mix until uniform.

### Preparation of Test Formulation B

### Chlorophyllin Serum

| Phase | Ingredient (INCI Name) | % wt/wt |
|---|---|---|
| A | Carbomer (2% Dispersion) | 55.00 |
| B | Butylene Glycol | 1.15 |
| B | Sodium Lactate | 1.60 |
| B | Pentylene Glycol | 4.00 |
| B | Phenoxyethanol | 0.47 |
| C | Purified Water | 26.76 |
| D | Vitamin E Acetate | 0.10 |
| D | Green Tea Extract | 0.10 |
| E | Sodium Hydroxide 32% | 1.64 |
| E | Purified Water | 8.58 |
| F | 5% Na Cu Chlorophyllin | 0.60 |

Phase A, the 2% dispersion of Carbomer, is prepared by mixing four components (each expressed as wt/wt % of the dispersion):

| Ingredient | % wt/wt |
|---|---|
| Carbomer | 2.00 |
| Butylene Glycol | 5.00 |
| Phenoxyethanol | 0.50 |
| Purified Water | 92.50 |

Add phenoxyethanol to butylene glycol; mix until clear and uniform. Add butylene glycol/phenoxyethanol mixture to water at room temperature; mix for 5 minutes. Disperse Carbomer (Acritamer 940; RITA Corporation) by slowly adding powder; mix at 600-2,000 rpm with a lightning type mixer for 60-90 minutes or until dispersion is smooth and uniform.

Prepare Phase B by mixing Phase B ingredients together with propeller until clear and uniform. Add Phase B to Phase A (2% dispersion of un-neutralized Carbomer) while mixing with propeller mixer until fully dispersed and uniform. Add Phase C to A/B; mix with propeller mixer until uniform. Add Phase D ingredients to A/B/C; mix for 5 minutes. Prepare Phase E by adding NaOH solution to water slowly with mixing. Add Phase E to A/B/C/D until fully dispersed. Measure pH of A/B/C/D/E; confirm pH is from about 7.2-7.6.

Phase F, the sodium copper chlorophyllin liposomal dispersion, is prepared as follows. (Constituent ingredients of the liposomal dispersion are listed based on their respective wt/wt percentages of the dispersion.)

| Ingredient | % wt/wt |
|---|---|
| Sodium Copper Chlorophyllin | 5.00 |
| Butylene Glycol | 5.00 |
| Phenoxyethanol | 0.5 |
| 30% Simethicone Emulsion USP | 0.005 |
| Lecithin fraction, enriched with phosphatidylcholine | 10.00 |
| Purified Water | 79.45 |

30% Simethicone Emulsion USP, a water-dilutable defoaming/antifoam agent containing 30% by weight of simethicone and non-ionic emulsifiers, is available under the trade name Dow Corning 7-9245.

The lecitihin fraction is preferably derived from soybean and is comprised of the following components (based on the total weight of the lecitihin fraction): phosphatidyl-choline at a concentration of at least about 85.0% wt/wt, phosphatidic acid at a concentration of from about 5% to about 7% wt/wt, and lysophosphatidylcholine at a concentration of up to about 3.0% wt/wt. A lecithin fraction meeting the above criteria is commercially available as Phosopholipon® 85G (Lipoid, Inc.).

Add Phase F (liposome dispersion) to A/B/C/D/E and mix until fully dispersed and uniform. (Set mixing speed to avoid cavitation and air entrapment.)

Study participants applied two test formulations—Retinol Lotion (0.25% or 0.5%) and Chlorophyllin Serum twice daily for an 8-week period. Prior to the study, baseline digital photographs were taken of each participant. Spectrophotometric intracutaneous analysis (SIA) of the concentration and distribution of one or more chromophores in the imaged areas of skin, including melanin and hemoglobin was utilized. The use of SIA to evaluate changes in melanin content is described in US Patent Application Publication 2007/0161910. US Patent Application Publication 2009/0080727 describes the use of SIA to determine the distribution of blood vessels in an imaged area of skin. Changes in skin texture and collagen thickness can also be assessed using SIA as described in US Patent Application Publications 2009/0080726 (measuring collagen thickness) and 2009/0043363 and 2008/0319283 (both relating to measuring skin texture). As summarized below, redness (denoted “a+”) and pigment levels (denoted “b/L”) measured by SIA.

Additionally, a self-administered patient survey was completed (by the patient) every 2 weeks, evaluating both the product's characteristics (ease of application) and its effects on the skin (redness, pore size, oiliness, smoothness, radiance, fine lines). Patients applied the serum twice daily (AM & PM). They were not to use any other skin-rejuvenating products or exfoliation products during the study.

Clinical Evaluation

An observer trained in clinical evaluation evaluated changes in the skin condition of study participants: according to the following parameters: evenness of skin tone; pore size and visible oiliness; dark circles and crepiness; pleating and coarse rhytids; nasolabial fold and marionette lines; fine lines; background erythema; background color; color of lentigines; skin laxity (in particular, skin laxity in relation to the definition of jaw line). In addition to visual examination, digital photographs were taken and compared at four week intervals (i.e. at baseline and at four and eight weeks after entering the study).

Clinical visual assessment was supplemented with objective measurement. Oiliness was measured using Sebutape. Sebutape measurements were taken on the right and left sides of the forehead as well as on the nose and the chin. These four measurements were summed and compared at baseline (start of the study) as well as at four weeks and eight weeks and are summarized in the charts below as number of patients assessed versus number of patients showing improvement, e.g., 11/15 (eleven of fifteen patients assessed showing improvement):

More Even Skin Tone

| | |
|---|---|
| 4 weeks | 8 weeks |

| Improvement | 0.25% | 0.5% | 0.25% | 0.5% |
|---|---|---|---|---|
| 0-24% | 11/11 | 11/15 | | 1/15 |
| 25-49% | | 4/15 | 10/11 | 12/15 |
| 50-74% | | | 1/11 | 2/15 |
| 75-100% | | | | |

Decrease in Pore Size/Oiliness

| | |
|---|---|
| 4 weeks | 8 weeks |

| Improvement | 0.25% | 0.5% | 0.25% | 0.5% |
|---|---|---|---|---|
| 0-24% | 11/11 | 15/15 | | 2/15 |
| 25-49% | | | 10/11 | *13/15 |
| 50-74% | | | 1/11 | |
| 75-100% | | | | |

Decrease in Dark Circles/Crepiness

| | |
|---|---|
| 4 weeks | 8 weeks |

| Improvement | 0.25% | 0.5% | 0.25% | 0.5% |
|---|---|---|---|---|
| 0-24% | 11/11 | 14/15 | 11/11 | 4/15 |
| 25-49% | | 1/15 | | 11/15 |
| 50-74% | | | | |
| 75-100% | | | | |

Decreased Pleating/Coarse Rhytids

| | |
|---|---|
| 4 weeks | 8 weeks |

| Improvement | 0.25% | 0.5% | 0.25% | 0.5% |
|---|---|---|---|---|
| 0-24% | 5/5 | 6/7 | | 3/7 |
| 25-49% | | 1/7 | 5/5 | 4/7 |
| 50-74% | | | | |
| 75-100% | | | | |

Decrease in Nasolabial Fold and Marionette Line

| | |
|---|---|
| 4 weeks | 8 weeks |

| Improvement | 0.25% | 0.5% | 0.25% | 0.5% |
|---|---|---|---|---|
| 0-24% | 7/7 | *12/12 | 2/7 | 11/12 |
| 25-49% | | | 5/7 | 1/12 |
| 50-74% | | | | |
| 75-100% | | | | |

Decrease in Fine Lines

| | |
|---|---|
| 4 weeks | 8 weeks |

| Improvement | 0.25% | 0.5% | 0.25% | 0.5% |
|---|---|---|---|---|
| 0-24% | 10/10 | 14/15 | | 8/15 |
| 25-49% | | 1/15 | 10/10 | 7/15 |
| 50-74% | | | | |
| 75-100% | | | | |

Decrease in Background Erythema

| | |
|---|---|
| 4 weeks | 8 weeks |

| Improvement | 0.25% | 0.5% | 0.25% | 0.5% |
|---|---|---|---|---|
| 0-24% | 11/11 | 14/15 | | 2/15 |
| 25-49% | | 1/15 | 11/11 | 9/15 |
| 50-74% | | | | 4/15 |
| 75-100% | | | | |

Decrease in Diameter of Blood Vessels

| | |
|---|---|
| 4 weeks | 8 weeks |

| Improvement | 0.25% | 0.5% | 0.25% | 0.5% |
|---|---|---|---|---|
| 0-24% | 11/11 | 15/15 | 7/11 | 8/15 |
| 25-49% | | | 4/11 | 7/15 |
| 50-74% | | | | |
| 75-100% | | | | |

Decrease in Background Pigment

| | |
|---|---|
| 4 weeks | 8 weeks |

| Improvement | 0.25% | 0.5% | 0.25% | 0.5% |
|---|---|---|---|---|
| 0-24% | 11/11 | 14/15 | 3/11 | 4/15 |
| 25-49% | | 1/15 | 8/11 | 10/15 |
| 50-74% | | | | 1/15 |
| 75-100% | | | | |

Decrease in Color of Lentigenes

| | |
|---|---|
| 4 weeks | 8 weeks |

| Improvement | 0.25% | 0.5% | 0.25% | 0.5% |
|---|---|---|---|---|
| 0-24% | 11/11 | 15/15 | 5/11 | 10/15 |
| 25-49% | | | 5/11 | 1/15 |
| 50-74% | | | 1/11 | |
| 75-100% | | | | |

More Defined Jawline

| | |
|---|---|
| 4 weeks | 8 weeks |

| Improvement | 0.25% | 0.5% | 0.25% | 0.5% |
|---|---|---|---|---|
| 0-24% | 10/10 | 12/12 | 10/10 | 11/12 |
| 25-49% | | | | 1/12 |
| 50-74% | | | | |
| 75-100% | | | | |

Decreased 3-5 a+ readings

| | |
|---|---|
| 4 weeks | 8 weeks |

| | | | | |
|---|---|---|---|---|
| Improvement | 0.25% | 0.5% | 0.25% | 0.5% |

| | | |
|---|---|---|
| 0-24% | 5/11 | 6/14 |
| 25-49% | | |
| 50-74% | | |
| 75-100% | | |

Decreased 3-5 b/L readings

| | |
|---|---|
| 4 weeks | 8 weeks |

| | | | | |
|---|---|---|---|---|
| Improvement | 0.25% | 0.5% | 0.25% | 0.5% |

| | | |
|---|---|---|
| 0-24% | 3/11 | 7/14 |
| 25-49% | | |
| 50-74% | | |
| 75-100% | | |

Subjective Assessment—0.25%

| | smoother, | | ↓ dark | ease of | | | |
|---|---|---|---|---|---|---|---|
| | ↑ radiance | ↓ pore size | circles | makeup | ↓ redness | ↓ pigment | ↓ fine lines |
| 2 weeks | 9/12 | 6/12 | 2/12 | 4/12 | 3/12 | 2/12 | 5/12 |
| 4 weeks | 10/12 | 5/12 | 3/12 | 6/12 | 7/12 | 6/12 | 4/12 |
| 6 weeks | 11/12 | 6/12 | 3/12 | 6/12 | 8/12 | 7/12 | 6/12 |
| 8 weeks | 11/12 | 8/12 | 3/12 | 7/12 | 10/12 | 8/12 | 8/12 |

Subjective Assessment—0.5%

| | smoother, | | ↓ dark | ease of | | | |
|---|---|---|---|---|---|---|---|
| | ↑ radiance | ↓ pore size | circles | makeup | ↓ redness | ↓ pigment | ↓ fine lines |
| 2 weeks | * 13/15 | * 5/15 | * 1/15 | * 5/15 | * 5/15 | * 3/15 | * 6/15 |
| 4 weeks | * 14/15 | * 7/15 | * 4/15 | * 6/15 | * 6/15 | * 8/15 | * 7/15 |
| 6 weeks | * 12/15 | * 8/15 | * 4/15 | * 7/15 | * 8/15 | * 6/15 | * 7/15 |
| 8 weeks | * 15/15 | * 11/15 | * 4/15 | * 9/15 | * 14/15 | * 9/15 | * 7/15 |
| 10 weeks | * 13/15 | * 10/15 | * 5/15 | * 9/15 | * 10/15 | * 9/15 | * 9/15 |

Another embodiment of the present invention is direction to a method for controlling, preventing the formation of and/or clearing visible open comedones (“blackheads”) or closed comedones (“whiteheads”) associated with acne vulgaris comprising administering a copper-chlorophyllin/vitamin C derivative/retinoid combination therapy. The combination therapy may be administered using a dual-chamber container.

A further embodiment of the present invention is directed to method of treatment of inflammatory dermatoses selected from the group consisting of inflammatory acne, erythematotelangiectaticrosacea, papulopustular rosacea, alopecia greata, and persistent seborrheic dermatitis. The method includes the step of topically administering to an area of skin affected by the inflammatory dermatosis a composition comprising (i) chlorophyllin, chlorin e4, chlorin e6, or ethyl esters of chlorin e4 or e6, the chlorins or chlorin ethyl esters preferably in the form of the sodium or potassium salts of their copper and zinc complexes, is administered together with (ii) vitamin C or a vitamin C derivative, preferably ascorbate, more preferably tetrahexyldecylascorbate.

As used in the present application, “inflammatory acne” describes a dermatologic condition in which multiple inflammatory lesions as well as several to many comedones and papules/pustules are present; there may or may not be small nodulo-cystic lesions.

As used in the present application, by the inflammatory dermatosis “erythemato-telangiectatic rosacea” is meant a subtype of rosacea characterized by flushing and persistent central facial erythema. Telangiectases are common in this subtype.

Rosacea can be, and preferably is graded by the standard grading system disclosed in Jonathan Wilkin, M D et al., J. Amer. Acad. Dermatology, 50(6), 907-12 (2004).

Acne can be and preferably is graded by the grading scale proposed in C. H. Cook et al., Arch. Dermatology, 571-575 (1979).

As used in the present application “papulopustular rosacea” is an inflammatory dermatosis characterized by persistent central facial erythema with transient papules, pustules, or both in a central facial distribution. The presentation of the erythemato-telangiectatic and papulopustular subtypes of rosacea are described in Wilkin et al., J. Am. Acad. Dermatol., pp. 907-912 (June 2004).

A further embodiment of the present invention provides a method for treating dermatologic conditions characterized by uneven pigmentation. The dermatologic condition is treated by a single formulation containing a copper chlorophyllin complex and a vitamin C derivative (sodium ascorbyl phosphate). Other objects and advantages of this aspect of the invention will become apparent and obvious from the following example, which is merely illustrative.

### Example 2

### Skin Lightener

| | | |
|---|---|---|
| A | Hydroxyethylcellulose - Natrasol HHR Solution (2%) | 40.000 |
| B | Butylene Glycol | 1.150 |
| B | Sodium Lactate | 1.600 |
| B | Diethylene Glycol Monoethyl Ether | 4.000 |
| | (Transcutol P available from Gattefosse) | |
| B | Phenoxyethanol | 0.465 |
| B | Purified Water | 24.00 |
| B | Tocopheryl Acetate | 0.100 |
| B | Sodium Ascorbyl Phosphate | 3.000 |
| C | Sodium Hydroxide 32% | 1.750 |
| C | Purified Water | 21.935 |
| D | 5% Na Cu Chlorophyllin | 2.000 |

Phase A (2% Natrosol HHR Solution) was formed by mixing 2% wt/wt hydroxyethylcellulose with 98% butylene glycol. Mixing was performed with a magnetic stirrer at room temperature. Mix Phase B ingredients using a propeller mixer at approximately 500 to 800 rpm for about 5 minutes, or until clear and uniform. Add Phase A to Phase B; mix with a propeller mixer at approximately 500 to 800 rpm for about 5 minutes, or until a clear, uniform solution has been achieved. Separately combine Phase C. While mixing with propeller mixer, slowly add the NaOH solution (Phase C) to NB. Confirm that pH of A/B/C is from about 7.2 to about 7.6. Add Phase D (liposomal dispersion formed in similar fashion to Example 1); mix with propeller mixer for 5 minutes at 1,000 rpm. Final product is a viscous dark green gel/serum.

### Example 3

### Chlorophyllin Gel 0.01% w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.7189 |
| Sodium Copper Chlorophyllin | 0.01 |
| 30% Simethicone Emulsion | 0.0001 |
| Lecithin 85G | 0.02 |

Ninety percent of the water is heated to 55° C. Using a mixer, carbomer is slowly added and mixed until uniformly dispersed. Five percent of the water is combined with the chlorophyllin and 5% with the Sodium hydroxide and set aside. Under continual mixing, each of the remaining ingredients are combined and mixed until uniform. The sodium hydroxide solution is slowly added until fully dispersed and uniform gel forms. Add chlorophyllin.

### Example 4

### Chlorophyllin Gel 0.001% w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.7279 |
| Sodium Copper Chlorophyllin | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the chlorophyllin and 5% with the Sodium hydroxide and set aside. Under continual mixing, each of the remaining ingredients are added and mixed until uniform. The sodium hydroxide solution is slowly added until fully dispersed and uniform a gel forms. Chlorophyllin solution is then combined.

### Example 5

### Chlorophyllin Gel 0.005% w/Na Ascorbate Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.7239 |
| Sodium Copper Chlorophyllin | 0.005 |
| 30% Simethicone Emulsion | 0.0001 |
| Lecithin 85G | 0.02 |

Ninety percent of the water is heated to 55° C. Using a mixer, slowly carbomer is slowly added and mixed until uniform and dispersed. Five percent of the water is mixed with the chlorophyllin and 5% with the Sodium hydroxide and set aside. Under continual mixing, each of the remaining ingredients are added and mixed until uniform. The sodium hydroxide solution is slowly added and mixed until fully dispersed and uniform gel forms. Chlorophyllin is then added.

### Example 6

### Chlorophyllin Gel 0.02% w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.7089 |
| Sodium Copper Chlorophyllin | 0.02 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is mixed with the chlorophyllin and 5% with the Sodium hydroxide and set aside. Under continual mixing, each of the remaining ingredients are slowly added and mixed until uniform. The sodium hydroxide solution is slowly added until fully dispersed and uniform gel forms. Chlorophyllin solution is then added.

### Example 7

### Chlorophyllin Gel 0.075% w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.6539 |
| Sodium Copper Chlorophyllin | 0.075 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is mixed with the chlorophyllin and 5% with the Sodium hydroxide and set aside. Under continual mixing, each of the remaining ingredients is added and mixed until uniform. The sodium hydroxide solution is slowly added and mixed until fully dispersed and uniform gel forms. Chlorophyllin solution is then added.

### Example 8

### Sodium Copper Chlorophyllin Gel 0.1% w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.4889 |
| Sodium Copper Chlorophyllin | 0.1 |
| 30% Simethicone Emulsion | 0.0001 |
| Lecithin 85G | 0.2 |

Ninety percent of the water is heated to 55° C. Using a mixer, carbomer is slowly added and mixed until uniform and dispersed. Five percent of the water is combined with the chlorophyllin and 5% with the Sodium hydroxide and set aside. Under continual mixing, each of the remaining ingredients are slowly added and mixed until uniform. The sodium hydroxide solution is slowly added and mixed until fully dispersed and uniform gel forms. The chlorophyllin solution is then added.

### Example 9

### Chloro Gel 0.01%—Sodium Copper Chlorin e4 Gel (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Sodium Copper Chlorin e4 | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is mixed with the porphyrin (Na Cu chlorin e4) and 5% with the sodium hydroxide and set aside. Under continual mixing, each of the remaining ingredients are added and mixed until uniform. The sodium hydroxide solution is slowly added until fully dispersed and uniform gel forms. The porphyrin solution is then added.

### Example 10

### Sodium Copper Chlorin e4 Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Sodium Copper Chlorin e4 | 0.001 |
| 30% Simethicone Emulsion | 0.0001 |
| Lecithin 85G | 0.02 |

Ninety of the water is heated to 55° C. Using a mixer, carbomer is slowly added and mixed until uniform and dispersed. Five percent of the water is combined with the porphyrin (Na Cu chlorin e4) and 5% with the sodium hydroxide and set aside. Under continual mixing, each of the remaining ingredients is added and mixed until uniform. The sodium hydroxide solution is slowly added and mixed until fully dispersed and uniform gel forms. Porphyrin solution is then added.

### Example 11

### Sodium Copper Chlorin e4 Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Sodium Copper Chlorin e4 | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 12

### Sodium Copper Chlorin e4 Gel 0.02%

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7239 |
| Sodium Copper Chlorin e4 | 0.02 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4) and 5% with the Sodium hydroxide and set aside. Under continual mixing, each of the remaining ingredients is added and mixed until uniform. The sodium hydroxide solution is slowly added and mixed until fully dispersed and uniform gel forms. The porphyrin solution is then added.

### Example 13

### Sodium Copper Chlorophyllin Gel (0.075%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.4889 |
| Sodium Copper Chlorin e4 | 0.075 |
| 30% Simethicone Emulsion | 0.0001 |
| Lecithin 85G | 0.2 |

Ninety percent of the water is heated to 55° C. Using a mixer, carbomer is slowly added and mixed until uniform and dispersed. Five percent of the water is mixed with the porphyrin (Na Cu chlorin e4) and 5% with the sodium hydroxide and set aside. Under continual mixing, each of the remaining ingredients is slowly added and mixed until uniform. The sodium hydroxide solution is slowly added and mixed until fully dispersed and uniform gel forms. The porphyrin solution is then added.

### Example 14

### Sodium Copper Chlorophyllin Gel (0.1%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.6439 |
| Sodium Copper Chlorin e4 | 0.1 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then added.

### Example 15

### Sodium Copper Chlorin e6 Gel (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Sodium Copper Chlorin e6 | 0.01 |
| 30% Simethicone Emulsion | 0.0001 |
| Lecithin 85G | 0.02 |

Ninety percent of the water to is heated to 55° C. Using a mixer, carbomer is slowly added and mixed until uniform and dispersed. Five percent of the water is combined with the porphyrin (Na Cu chlorin e6) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then added.

### Example 16

### Sodium Copper Chlorin e6 Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Sodium Copper Chlorin e6 | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e6) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then added.

### Example 17

### Sodium Copper Chlorin Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Sodium Copper Chlorin e6 | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e6) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then added.

### Example 18

### Sodium Copper Chlorin e6 Gel (0.02%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7239 |
| Sodium Copper Chlorin e6 | 0.02 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e6) and 5% with the sodium hydroxide and set aside. Under continual mixing, each of the remaining ingredients is added and mixed until uniform. The sodium hydroxide solution slowly is slowly added and mixed until fully dispersed and uniform gel forms. The porphyrin solution is then added.

### Example 19

### Sodium Copper Chlorin e6 Gel (0.075%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.6689 |
| Sodium Copper Chlorin e6 | 0.075 |
| 30% Simethicone Emulsion | 0.0001 |
| Lecithin 85G | 0.02 |

Ninety percent of the water is heated to 55° C. Using a mixer, carbomer is added and mixed until uniform and dispersed. Five percent of the water is combined with the porphyrin (Na Cu chlorin e6) and 5% with the sodium hydroxide and set aside. Under continual mixing, each of the remaining ingredients is added and mixed until uniform. The sodium hydroxide solution is slowly added until fully dispersed and uniform gel forms. The porphyrin solution is then added.

### Example 20

### Sodium Copper Chlorin e6 Gel (0.1%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.6439 |
| Sodium Copper Chlorin e6 | 0.1 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e6) and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then added.

### Example 21

### Sodium Copper Chlorin e4 Ethyl Ester (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Sodium Copper Chlorin e4 ethyl ester | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4 ethyl ester) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 22

### Sodium Copper Chlorin e4 Ethyl Ester Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Sodium Copper Chlorin e4 ethyl ester | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4 ethyl ester) and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 23

### Sodium Copper Chlorin e4 Ethyl Ester Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Sodium Copper Chlorin e4 ethyl ester | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e4) and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 24

### Sodium Copper Chlorin e4 Ethyl Ester Gel (0.02%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7239 |
| Sodium Copper Chlorin e4 ethyl ester | 0.02 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 25

### Sodium Copper Chlorin e4 Ethyl Ester Gel (0.075%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.4889 |
| Sodium Copper Chlorin e4 ethyl ester | 0.075 |
| 30% Simethicone Emulsion | 0.0001 |
| Lecithin 85G | 0.2 |

Ninety percent of the water is heated to 55° C. Using a mixer, carbomer is slowly added and mixed until uniform and dispersed. Five percent of the water is combined with the porphyrin and 5% with the sodium hydroxide and set aside. Under continual mixing, each of the remaining ingredients is added and mixed until uniform. The sodium hydroxide is solution slowly added and mixed until fully dispersed and uniform gel forms. The porphyrin solution is then combined.

### Example 26

### Sodium Copper Chlorin e4 Ethyl Ester Gel (0.1%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.4639 |
| Sodium Copper Chlorin e4 ethyl ester | 0.1 |
| 30% Simethicone Emulsion | 0.0001 |
| Lecithin 85G | 0.2 |

Ninety percent of the water is heated to 55° C. Using a mixer, carbomer is slowly added and mixed until uniform and dispersed. Five percent of the water is combined with the porphyrin (Na Cu chlorin e4 ethyl ester) and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 27

### Sodium Copper Chlorin e6 Ethyl Ester (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Sodium Copper Chlorin e6 ethyl ester | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Cu chlorin e6 ethyl ester) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 28

### Sodium Copper Chlorin e6 Ethyl Ester Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Sodium Copper Chlorin e6 ethyl ester | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin and 5% with the sodium hydroxide and set aside. Under continual mixing, each of the remaining ingredients are slowly added and mixed until uniform. The sodium hydroxide solution is slowly added and mixed until fully dispersed and uniform gel forms. The porphyrin solution is then combined.

### Example 29

### Sodium Copper Chlorin e6 Ethyl Ester Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Sodium Copper Chlorin e6 ethyl ester | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 30

### Sodium Copper Chlorin e6 Ethyl Ester Gel (0.02%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7239 |
| Sodium Copper Chlorin e6 ethyl ester | 0.02 |
| 30% Simethicone Emulsion | 0.0001 |
| Lecithin 85G | 0.02 |

Ninety percent of the water is heated to 55° C. Using a mixer, carbomer is slowly added and mixed uniform and dispersed. Five percent of the water is combined with the porphyrin and 5% with the sodium hydroxide and set aside. Under continual mixing, each of the remaining ingredients are slowly added and mixed until uniform. The sodium hydroxide solution is slowly added and mixed until fully dispersed and uniform gel forms. The porphyrin solution is then combined.

### Example 31

### Sodium Copper Chlorin e6 Ethyl Ester Gel (0.075%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.4889 |
| Sodium Copper Chlorin e6 ethyl ester | 0.075 |
| 30% Simethicone Emulsion | 0.0001 |
| Lecithin 85G | 0.2 |

Ninety percent of the water is heated to 55° C. Using a mixer, carbomer is slowly added and mixed until uniform and dispersed. Five percent of the water is combined with the porphyrin (Na Cu chlorin e6 ethyl ester) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 32

### Sodium Copper Chlorin e6 Ethyl Ester Gel (0.1%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.4639 |
| Sodium Copper Chlorin e6 ethyl ester | 0.1 |
| 30% Simethicone Emulsion | 0.0001 |

### Lecithin 85G 0.2

Ninety percent of the water is heated to 55° C. Using a mixer, carbomer is slowly added and mixed until uniform and dispersed. Five percent of the water are combined with the porphyrin and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 33

### Potassium Copper Chlorpophyllin Gel (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Potassium Copper Chlorophyllin | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the chlorophyllin (K Cu chlorophyllin) and 5% with the sodium hydroxide and set aside. Under continual mixing, each of the remaining ingredients are added and mixed until uniform. The sodium hydroxide solution is slowly added and mixed until fully dispersed and uniform gel forms. The solution of potassium copper chlorophyllin is then combined.

### Example 34

### Potassium Copper Chlorin e4 Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Potassium Copper Chlorin e4 | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (K Cu chlorin e4) and 5% with the sodium hydroxide and set aside. Under continual mixing, each of the remaining ingredients is added and mixed until uniform. The sodium hydroxide solution is slowly added and mixed until fully dispersed and uniform gel forms. The porphyrin solution is then combined.

### Example 35

### Potassium Copper Chlorin e6 Ethyl Ester Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Potassium Copper Chlorin e6 ethyl ester | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (K Cu chlorin e6 ethyl ester) and 5% with the sodium hydroxide and set aside. Under continual mixing, each of the remaining ingredients are slowly added and mixed until uniform. The sodium hydroxide solution is slowly added and mixed until fully dispersed and uniform gel forms. The porphyrin solution is then combined.

### Example 36

### Potassium Zinc Chlorpophyllin Gel (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Potassium Zinc Chlorophyllin | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the chlorophyllin and 5% with the odium hydroxide and set aside. Under continual mixing, each of the remaining ingredients are added and mixed until uniform. The sodium hydroxide solution is slowly added and mixed until fully dispersed and uniform gel forms. The chlorophyllin solution is then combined.

### Example 37

### Potassium Zinc Chlorin e4 Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Potassium Zinc Chlorin e4 | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (K Zn chlorin e4) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 38

### Potassium Zinc Chlorin e6 Ethyl Ester Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Potassium Zinc Chlorin e6 ethyl ester | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (K Zn chlorin e6 ethyl ester) and 5% with the sodium hydroxide and set aside. Under continual mixing, each of the remaining ingredients are added and mixed until uniform. The sodium hydroxide is solution slowly added and mixed until fully dispersed and uniform gel forms. The porphyrin solution is then combined.

### Example 39

### Sodium Zinc Chlorophyllin Gel (0.01%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7339 |
| Sodium Zinc Chlorophyllin | 0.01 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the chlorophyllin and 5% with the sodium hydroxide and set aside. Under continual mixing, each of the remaining ingredients are added and mixed until uniform. The sodium hydroxide solution is slowly added and mixed until fully dispersed and uniform gel forms. The chlorophyllin solution is then combined.

### Example 40

### Sodium Zinc Chlorin e4 Gel (0.001%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7429 |
| Sodium Zinc Chlorin e4 | 0.001 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Zn chlorin e4) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 41

### Sodium Zinc Chlorin e6 Ethyl Ester Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Sodium Zinc Chlorin e6 ethyl ester | 0.005 |
| 30% Simethicone Emulsion | 0.0001 |
| Lecithin 85G | 0.02 |

Ninety percent of the water is heated to 55° C. Using a mixer, carbomer is slowly added and mixed until uniform and dispersed. Five percent of the water is combined with the porphyrin (Na Zn chlorin e6 ethyl ester) and 5% with the sodium hydroxide and set aside. Under continual mixing, each of the remaining ingredients are added and mixed until uniform. The sodium hydroxide solution is slowly added and mixed until fully dispersed and uniform gel forms. The porphyrin solution is then combined.

### Example 42

### Sodium Copper Chlorophyllin Liposome Gel (0.01%) w/Na Ascorbyl Phosphate

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Sodium Ascorbyl Phosphate | 0.025 |
| Purified Water | 86.7189 |
| Sodium Copper Chlorophyllin | 0.01 |
| 30% Simethicone Emulsion | 0.0001 |
| Lecithin 85G | 0.02 |

A chlorophyllin liposome is prepared by hydrating the lecithin into 20% of the water in the formula for an hour. The chlorophyllin is slowly added into an additional 20% of the water separately, slowly heated to 65° C. while mixing with a propeller mixer. When fully dispersed, the mixture is transferred to a homogenizer and homogenized at 5000 RPM during which time hydrated lecithin mixture is slowly added. Homogenization is continued at 7000 RPM for 10 minutes.

As a separate preparation, the carbomer is slowly added into the remaining water while heating to 55° C. A propeller mixer is used to create a vortex and the carbomer powder is slowly introduced into the vortex. Stirrer speed is gradually increased as the solution thickens. Mixing is continued for approximately one hour until the carbomer is fully hydrated and dispersed. The remaining ingredients, except the sodium hydroxide, are introduced into this dispersion and mixed until uniform. The sodium hydroxide solution is slowly added while mixing to create a clear and uniform gel. The prepared liposomes are carefully added and mixed until fully dispersed.

### Example 43

### Potassium Zinc Chlorin e6 Ethyl Ester Liposome Gel (0.005%)

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Potassium Zinc Chlorin e6 ethyl ester | 0.005 |
| 30% Simethicone Emulsion | 0.0001 |
| Lecithin 85G | 0.02 |

A porphyrin (Zn chlorin e6 ethyl ester) liposome is created by hydrating the lecithin into 20% of the water in the formula for an hour. The porphyrin is slowly added into an additional 20% of the water separately, slowly heated to 65 C while mixing with a propeller mixer. When fully dispersed, the mixture is transferred to a homogenizer and homogenized at 5000 RPM during which time the hydrated lecithin mixture is slowly added. Homogenization is continued at 7000 RPM for 10 minutes.

As a separate preparation, the carbomer is slowly added into the remaining water while heating to 55 C. A vortex is created with a propeller mixer and the carbomer powder is slowly introduced into the vortex, the speed of the propeller mixer is increased as the solution thickens. The combination is mixed for approximately one hour until the carbomer is fully hydrated and dispersed. The remaining ingredients, minus the Sodium Hydroxide solution, are added to this dispersion that is mixed until uniform. The sodium hydroxide solution is slowly added while mixing to obtain a clear and uniform gel. The prepared liposome dispersion is added and mixed until fully dispersed.

### Example 44

### Potassium Copper Chlorin e6 Ethyl Ester Liposome Gel 0.005%

| Ingredient | % w/w |
|---|---|
| Carbomer | 1.1 |
| 1,3-Butylene Glycol | 3.91 |
| Sodium Lactate, 60% | 1.6 |
| Pentylene Glycol | 4.0 |
| Phenoxyethanol | 1.026 |
| Sodium Hydroxide, 33% | 1.59 |
| Vitamin E Acetate | 0.01 |
| Purified Water | 86.7389 |
| Potassium Copper Chlorin e6 ethyl ester | 0.005 |
| 30% Simethicone Emulsion | 0.0001 |
| Lecithin 85G | 0.02 |

A porphyrin (chlorine e6 ethyl ester salts) liposome is prepared by hydrating the lecithin into 20% of the water of the formula for an hour. The porphyin is slowly added into an additional 20% of the water, separately, and is slowly heated to 65° C. while mixing with a propeller mixer. When fully dispersed, the combination so obtained is transferred to a vessel with a homogenizer and homogenized at 5000 RPM, during which time the hydrated lecithin mixture is slowly introduced to the homogenizer. Homogenization is then continued at 7000 RPM for 10 minutes.

As a separate preparation, the carbomer is slowly added into the remaining water while being heated to 55° C. A vortex is created in the mixture with a propeller mixer and the carbomer powder is slowly added into the vortex slowly, increasing speed as the solution thickens. The combination is mixed for approximately one hour until the carbomer is fully hydrated and dispersed. The remaining ingredients, minus the sodium hydroxide solution, are added to this dispersion and mixed until uniform. The sodium hydroxide solution is slowly added to the mixer with uniform agitation to obtain a clear and uniform gel. The prepared liposome dispersion is then combined with the mixture until the liposomes are fully dispersed.

### Example 45

### Retinol Lotion with Vitamin C

| Phase | Ingredient | % w/w |
|---|---|---|
| A | Purified Water | 71.65 |
| A | Carbomer | 0.500 |
| A | Xanthan Gum | 0.200 |
| A | Disodium EDTA | 0.100 |
| B | Ethylhexyl Stearate | 4.000 |
| B | Glyceryl Stearate, PEG-100 Stearate | 4.000 |
| B | Cetearyl Alcohol, Steareth-10, Steareth-20 | 2.000 |
| B | Caprylic/Capric Triglyceride | 5.000 |
| B | Dimethicone | 1.000 |
| B | PPG-12/SMDI Copolymer | 0.500 |
| B | Polyacrylamide, C13-C14 Isoparaffin, Laureth-7 | 1.000 |
| C | Phenoxyethanol | 0.800 |
| C | Tocopherol Acetate | 0.500 |
| C | Glycerin, Palmitoyl Tripeptide-5 | 3.000 |
| C | Tetrahexyldecyl Ascorbate | 0.500 |
| D | Isopentyldiol | 4.000 |
| D | Allyl Methacrylate Crosspolymer, Polysorbate 20, | 1.250 |
| | Retinol, BHT | |

Phase A is assembled by first combining carbomer and xanthan gum. The combination is slowly added to the rest of Phase A while mixing with a propeller mixer. Phase A is heated to 55° C. Phase B ingredients are combined and heated heat to 55° C. Phases C and D are separately assembled. Phase A is slowly added into Phase B and immediately homogenized with a Silverson L4RT homogenizer with standard head at 7,000 rpm for 5-10 minutes. Phase C is added to NB at room temperature (20-25° C.) and the resulting mixture homogenized with Silverson homogenizer at 3,000 rpm for about 2 minutes or until uniform. Phase D is added to thus combined phases A/B/C at room temperature and the resulting final combination mixed until uniform.

### Example 46

### Chlorophyllin Lotion

| Phase | Ingredient | % w/w |
|---|---|---|
| A | Caprylic/Capric Triglyceride | 4.000 |
| A | Cetyl Alcohol | 2.000 |
| A | Stearyl Alcohol | 2.000 |
| A | Glyceryl Stearate and PEG-100 Stearate | 3.000 |
| B | Glycerin | 5.000 |
| B | Pentylene Glycol | 3.000 |
| B | Butylene Glycol | 2.010 |
| B | Glycereth-5 Lactate, Lactic Acid, Clycereth-5 | 5.000 |
| B | Phenoxyethanol | 0.676 |
| B | Carbomer | 0.800 |
| B | Purified Water | 68.9839 |
| B | Sodium Lactate and Water | 2.000 |
| B | Triethanolamine | 1.000 |
| C | Lactic Acid | 0.500 |
| C | Sodium Copper Chlorophyllin | 0.010 |
| C | Lecithin 85G | 0.020 |
| C | 30% Simethicone Emulsion | .0001 |

Components of Phase A are combined and heated to 55° C. Water and carbomer are combined until uniform dispersion forms. The rest of phase B ingredients are combined and the combination heated heat to 55 C. Phase C ingredients are combined. Under a Silverson Homoginizer, Phase B is mixed into Phase A, mixing at around 5000 RPM. Phase C is slowly added. The resulting mixture is mixed at 9000 RPM to obtain a uniform lotion results.

### Example 47

### Potassium Copper Chlorin e6 Ethyl Ester Lotion

| Phase | Ingredient | % w/w |
|---|---|---|
| A | Caprylic/Capric Triglyceride | 4.000 |
| A | Cetyl Alcohol | 2.000 |
| A | Stearyl Alcohol | 2.000 |
| A | Glyceryl Stearate and PEG-100 Stearate | 3.000 |
| B | Glycerin | 5.000 |
| B | Pentylene Glycol | 3.000 |
| B | Butylene Glycol | 2.010 |
| B | Glycereth-5 Lactate, Lactic Acid, Clycereth-5 | 5.000 |
| B | Phenoxyethanol | 0.676 |
| B | Carbomer | 0.800 |
| B | Purified Water | 68.9839 |
| B | Sodium Lactate and Water | 2.000 |
| B | Triethanolamine | 1.000 |
| C | Lactic Acid | 0.500 |
| C | Potassium Copper Chlorin e6 ethyl ester | 0.010 |
| C | Lecithin 85G | 0.020 |
| C | 30% Simethicone Emulsion | .0001 |

Components of Phase A are combined and heated to 55° C. Water and carbomer are combined and mixed until a uniform dispersion forms, to which dispersion the rest of phase B ingredients are added and the resulting mixture heated to 55° C. The Phase C ingredients are combined. Under a Silverson Homoginizer, Phase B is mixed into Phase A, mixing at around 5000 RPM. Phase C is slowly added to the resulting mixture and mixing at 9000 RPM is continued until a uniform lotion is obtained.

### Example 48

### Sodium Copper Chlorin e4 Lotion

| Phase | Ingredient | % w/w |
|---|---|---|
| A | Caprylic/Capric Triglyceride | 4.000 |
| A | Cetyl Alcohol | 2.000 |
| A | Stearyl Alcohol | 2.000 |
| A | Glyceryl Stearate and PEG-100 Stearate | 3.000 |
| B | Glycerin | 5.000 |
| B | Pentylene Glycol | 3.000 |
| B | Butylene Glycol | 2.010 |
| B | Glycereth-5 Lactate, Lactic Acid, Clycereth-5 | 5.000 |
| B | Phenoxyethanol | 0.676 |
| B | Carbomer | 0.800 |
| B | Purified Water | 68.9839 |
| B | Sodium Lactate and Water | 2.000 |
| B | Triethanolamine | 1.000 |
| C | Lactic Acid | 0.500 |
| C | Sodium Copper Chlorin e4 | 0.010 |
| C | Lecithin 85G | 0.020 |
| C | 30% Simethicone Emulsion | .0001 |

The ingredients of Phase A are combined and heated to 55° C. Water and carbomer are combined until uniform dispersion forms, to which dispersion the rest of phase B ingredients are added and the resulting combination heated to 55 C. The ingredients of Phase C are combined. Under a Silverson Homoginizer, Phase B is mixed into Phase A, mixing at around 5000 RPM.

Phase C is slowly added to the resulting mixture. Mixing at 9000 RPM is continued until a uniform lotion is obtained.

### Example 50

### Efficacy in Treating Acne

Twenty patients having facial acne, with 20 to 50 inflammatory lesions (ILs) and 30 to 100 non-inflammatory lesions (NILs), are enrolled in an eight-week clinical study. By the term inflammatory lesions are meant papules, pustules and nodules. By the term non-inflammatory lesions is meant open and closed comedones.

Efficacy of treatment with the compositions of the present invention is measured at baseline and two, four and eight-week intervals from commencement of the study. More particularly, treatment success is measured based on the following criteria: skin appearing “clear” or “almost clear” based on dermatologist evaluation (i.e., clinical grading); reduction in ILs, NILs, and total lesion count; and patient self-assessment of acne improvement. Clinical grading assesses the degree of enlarged facial pores, oiliness and blotchiness. Additionally, oiliness is measured using Sebutape.

The majority of subjects (8/10) report improvement in their own skin condition in terms of reduced oiliness, decreased visibility of pores, and improved evenness of color and texture. Digital photographic analysis utilizing the VISIA® clinical grading system shows significant reduction in pore size and improved overall skin evenness.

1. A method for improving the cosmetic appearance of the skin by reducing one or more cosmetic dermatologic parameters selected from the group of fine lines, coarse wrinkles, pleating, marionette lines, nasolabial folds, pore size, oiliness, dark circles, uneven pigmentation, and redness comprising administering safe and effective amounts of
(a) a porphyrin selected from the group consisting of
(i) chlorin e4
(ii) sodium and potassium salts of copper complexes of chlorin e4
(iii) sodium and potassium salts of zinc complexes of chlorin e4
(b) vitamin C or a vitamin C derivative, and
(c) optionally, a retinoid.

2. A method for improving the cosmetic appearance of the skin by reducing one or more cosmetic dermatologic parameters selected from the group of fine lines, coarse wrinkles, pleating, marionette lines, nasolabial folds, pore size, oiliness, dark circles, uneven pigmentation, and redness comprising administering safe and effective amounts of
(a) a porphyrin selected from the group consisting of
(i) chlorin e6
(ii) sodium and potassium salts of copper complexes of chlorin e6
(iii) sodium and potassium salts of zinc complexes of chlorin e6
(b) vitamin C or a vitamin C derivative, and
(c) optionally, a retinoid.

3. A method for improving the cosmetic appearance of the skin by reducing one or more cosmetic dermatologic parameters selected from the group of fine lines, coarse wrinkles, pleating, marionette lines, nasolabial folds, pore size, oiliness, dark circles, uneven pigmentation, and redness comprising administering safe and effective amounts of
(a) a porphyrin selected from the group consisting of
(i) chlorin e4 ethyl ester
(ii) sodium and potassium salts of copper complexes of chlorin e4 ethyl ester
(iii) sodium and potassium salts of zinc complexes of chlorin e4 ethyl ester
(b) vitamin C or a vitamin C derivative, and
(c) optionally, a retinoid.

4. A method for improving the cosmetic appearance of the skin by reducing one or more cosmetic dermatologic parameters selected from the group of fine lines, coarse wrinkles, pleating, marionette lines, nasolabial folds, pore size, oiliness, dark circles, uneven pigmentation, and redness comprising administering safe and effective amounts of
(a) a porphyrin selected from the group consisting of
(i) chlorin e6 ethyl ester
(ii) sodium and potassium salts of copper complexes of chlorin e6 ethyl ester
(iii) sodium and potassium salts of zinc complexes of chlorin e6 ethyl ester
(b) vitamin C or a vitamin C derivative, and
(c) optionally, a retinoid.

5. A method for improving the cosmetic appearance of the skin by reducing one or more cosmetic dermatologic parameters selected from the group of fine lines, coarse wrinkles, pleating, marionette lines, nasolabial folds, pore size, oiliness, dark circles, uneven pigmentation, and redness comprising administering safe and effective amounts of
(a) a porphyrin selected from the group consisting of
(i) copper chlorophyllin
(ii) zinc chlorophyllin
(iii) sodium and potassium salts of copper chlorophyllin
(iv) sodium and potassium salts of zinc chlorophyllin
(b) vitamin C or a vitamin C derivative, and
(c) optionally, a retinoid.

6. (canceled)

7. (canceled)
